(19) United States
(12) Patent Application Publication
(10) Pub. No.: US 2022/0175800 A1
(43) Pub. Date: Jun. 9, 2022

(54) DISPERSIBLE TABLET COMPOSITION

(71) Applicant: PTC THERAPEUTICS INC., South Plainfield, NJ (US)

(72) Inventors: Akm Nasir Uddin, Somerset, NJ (US); Mandar Vasant Dali, Bridgewater, NJ (US)

(21) Appl. No.: 17/602,934

(22) PCT Filed: Apr. 10, 2020
(86) PCT No.: PCT/US2020/027717

§ 371 Date: Oct. 11, 2021

Related U.S. Application Data
(60) Provisional application No. 62/833,632, filed on Apr. 12, 2019.

(51) Int. Cl. A61K 31/58; A61K 9/20; A61K 47/26

(52) U.S. Cl. CPC A61K 31/58; A61K 9/2018; A61K 9/2054; A61K 47/26; A61K 9/2013; A61K 9/2009; A61K 9/2059

(57) ABSTRACT

The present description generally relates to a dispersible tablet composition of an active pharmaceutical ingredient (API), which rapidly disperses in water to provide a homogeneous dispersion that ensures uniformity of dose.

### CROSS-REFERENCE TO RELATED APPLICATIONS

This application claims priority to U.S. Provisional Application No. 62/833,632, filed Apr. 12, 2019, the contents of which are incorporated by reference herein.

### FIELD

The present description generally relates to a dispersible tablet composition of an active pharmaceutical ingredient (API), which rapidly disperses in water to provide a homogeneous dispersion that ensures uniformity of dose.

### BACKGROUND

Pharmaceutical suspensions are dispersions of particles of active pharmaceutical ingredients and excipients in a vehicle in which the API has minimum solubility. Such suspensions are usually formulated to improve chemical stability of the API and to mask any unpleasant taste. Such dosage forms have advantages over solid dosage forms, such as tablets, in that they are easier to swallow, particularly for small children. However, suspension as a dosage form is associated with issues such as microbial growth, sedimentation, and non-uniformity of dose. Antibacterial agents are typically included in suspension formulations. However, such agents can raise toxicity issues, particularly when administered to small children. Administration of suspensions to small children also requires provision of a special oral syringe, and care must be used when using such a device to ensure proper measurement and dosing. A syringe must also be carefully cleaned after use, if it is to be reused.

Solid dosage forms ensure uniformity of dosing, are more robust, and have fewer microbiological issues compared to liquid dosing forms. However, such dosage forms can be difficult for small children or older adults to swallow. Thus, there is a need for a composition which overcomes the problems associated with the swallowing of solid dosage forms but which provides a viable substitute for suspensions. One such dosage form is a dispersible tablet.

Dispersible tablet dosage forms provide advantages of both solid dosage forms and suspension formulations. Dispersible tablets are uncoated or film coated tablets designed to be dispersed in water before administration. Unlike suspension formulations, they can be provided without preservatives, and do not require a syringe to ensure correct dosing.

### SUMMARY

In one aspect described herein, a dispersible tablet of deflazacort is provided. The chemical name of deflazacort is (11β,16β)-21-(acetyloxy)11-hydroxy-2′-methyl-5′H-pregna-1,4-dieno[17,16-d]oxazole-3,20-dione, a compound having the following structure:

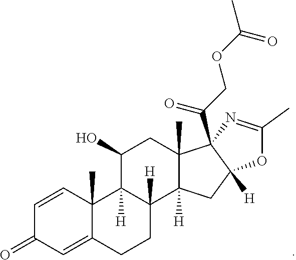

In another aspect described herein, the dispersible tablet comprises deflazacort, a water-soluble diluent, a water swellable diluent and a superdisintegrant, wherein the tablet disintegrates within 3 minutes when added to water at 20 to 25° C. In another aspect the dispersible tablet further comprises a lubricant. In another aspect the dispersible tablet further comprises a glidant. In another aspect the dispersible tablet further comprises a sweetener. In another aspect the dispersible tablet further comprises a flavoring agent.

In one aspect described herein, a process for preparing a dispersible tablet of deflazacort comprising blending a composition comprising the deflazacort, the water-soluble diluent, the water swellable diluent, superdisintegrant, lubricant, and glidant to form an admixture, and compressing the admixture to form a tablet. In another aspect, the composition further comprises a sweetener. In another aspect the composition further comprises a flavoring agent.

In one aspect described herein, a method of treating a form of muscular dystrophy is provided, comprising dispersing at least one dispersible tablet of deflazacort in water and administering the resulting suspension to a subject in need thereof. In another aspect described herein the form of muscular dystrophy is selected from Duchenne muscular dystrophy (DMD) and Limb-girdle muscular dystrophy (LGMD). The type of Duchenne muscular dystrophy treated can be Becker muscular dystrophy. The type of Limb-girdle muscular dystrophy treated can be Type 2I.

### DETAILED DESCRIPTION

In one aspect described herein, a dispersible tablet comprising deflazacort is provided. The chemical name of deflazacort is (11β,16β)-21-(acetyloxy)11-hydroxy-2′-methyl-5′H-pregna-1,4-dieno[17,16-d]oxazole-3,20-dione, a compound having the following structure:

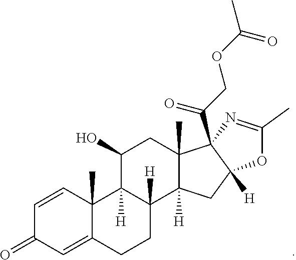

The dispersible tablet comprises deflazacort a water-soluble diluent, a water swellable diluent, a superdisintegrant, a lubricant, and a glidant, wherein the dispersible tablet provided is capable of dispersing in water in less than 3 minutes at about 25° C. In another aspect, the dispersible tablet is capable of dispersing in water in less than 1 minute.

In one aspect described herein, a process for preparing a dispersible tablet of deflazacort is provided, comprising blending a composition comprising deflazacort, a water-soluble diluent, a water swellable diluent, a superdisintegrant, a lubricant and a glidant to form an admixture, and compressing the admixture to form a tablet. In another aspect, the composition further comprises a sweetener. In another aspect the composition further comprises a flavoring agent. In another aspect, the process comprises preparing an initial blend of the deflazacort, the glidant, the water swellable diluent and some of the water soluble diluent, and mixing that before adding the remaining excipients to form the admixture that is compressed to form tablets.

In one aspect described herein, a method of treating a form of muscular dystrophy is provided, comprising dispersing at least one dispersible tablet of deflazacort in water and administering the resulting suspension to a subject in need thereof. In another aspect described herein the form of muscular dystrophy is selected from Duchenne muscular dystrophy (DMD) and Limb-girdle muscular dystrophy (LGMD). The Duchenne muscular dystrophy treated can be Becker muscular dystrophy (BMD), a type of Duchenne muscular dystrophy. The type of Duchenne muscular dystrophy treated can be Becker muscular dystrophy. The type of Limb-girdle muscular dystrophy treated can be Type 2I.

Deflazacort is a glucocorticoid with anti-inflammatory and immunosuppressive effects. It has been approved for use in treating Duchenne muscular dystrophy (DMD). DMD is a recessive X-linked form of muscular dystrophy which results in muscle degeneration, difficulty walking, breathing, and eventually death. The incidence is approximately 1 in every 3500 live male births. Deflazacort has also been used to treat other indications, including Limb-girdle muscular dystrophy. Limb-girdle muscular dystrophies (LGMDs), a heterogenous group of rare genetic myopathies with many subtypes categorized by disease, gene, and inheritance, are classified into two main groups (type 1 [autosomal dominant] and type 2 [autosomal recessive]. Limb-girdle muscular dystrophy type 2I, one of the more common forms of LGMD, a subtype of type 2 LGMD affects about 400 individuals in the US. Deflazacort is available commercially, under the brand name EMFLAZA® (PTC Therapeutics, Inc.). Symptoms of DMD usually appear in male children between the ages of 3 and 5 years, while the age of onset of LGMD2I ranges from 2 to 50 years. As noted above, the dispersible deflazacort tablets provided herein are particularly suitable for administration to young children, who cannot tolerate preservatives included in suspension formulations and can have difficulty swallowing solid dosage forms.

The deflazacort in the dispersible tablet compositions described herein is preferably present in micronized form. 90% of the micronized deflazacort particles are less than about 100 μm in size, preferably less than about 50 μM, more preferably less than about 10 μm in size, more preferably less than about 5 μm in size. 99% of the micronized deflazacort particles are preferably less than about 10 μM in size. Deflazacort is poorly soluble in water. Micronization enhances bioavailability and enhances dispersibility when the tablet is placed in water. The deflazacort is about 1 to about 20%, preferably about 2 to about 10% by weight of the tablet composition. The amount of deflazacort in each tablet depends upon the tablet size. Preferred dosages include 6, 18, 30, and 36 mg per tablet. The recommended dosage of deflazacort for adults and children is currently 0.9 mg/Kg.

The dispersible tablet compositions described herein include both water soluble and water swellable diluents. Suitable water-soluble diluents illustratively include, either individually or in combination, mannitol, lactose, xylitol, sorbitol, calcium sulfate dihydrate, inositol, dextrin, calcium sulfate anhydrate, fructose, kaolin, sucrose, lactitol, dextrates, sodium chloride, dextrose. Mannitol is particularly preferred. Such water-soluble diluents constitute in total about 10% to about 60%, preferably about 20% to about 50%, still more preferably about 25% to about 45% of the total weight of the composition.

Suitable water swellable diluents include, either individually or in combination, microcrystalline cellulose, pregelatinized starch, starch, powdered cellulose, silicified microcrystalline cellulose, dibasic calcium phosphate dihydrate, calcium phosphate, calcium carbonate, hydroxypropylcellulose, hydroxyethylcellulose, and hydroxypropyl methylcellulose. Microcrystalline cellulose is particularly preferred. Such water swellable diluents, if present, constitute in total of about 10% to about 70%, preferably about 20% to about 60%, more preferably about 30% to about 55% of the total weight of the composition.

The ratio of water swellable diluent(s) to water-soluble diluent(s) is preferably about 0.5 to about 1.8, more preferably about 0.6 to about 1.7, more preferably about 1.2 to about 1.65.

The dispersible tablet compositions described herein comprise at least one pharmaceutically acceptable superdisintegrant. Superdisintegrants ensure the rapid dispersiblity of the composition. Suitable superdisintegrants include, either individually or in combination croscarmellose sodium, sodium starch glycolate, and crospovidone. Croscarmellose sodium, sodium starch glycolate, or a combination of the two are preferred. Use of sodium starch glycolate as the superdisintegrant is particularly preferred. The superdisintegrant(s) may be present in an amount ranging from about 0.5% to about 30%, preferably about 1% to about 25%, more preferably about 5% to about 20% of the total weight of the composition.

In one aspect described herein, compositions optionally comprise one or more pharmaceutically acceptable lubricants and/or glidants as excipients. Suitable lubricants include, either individually or in combination stearic acid and salts thereof, including magnesium, calcium and sodium stearates; colloidal silica; waxes; boric acid; sodium benzoate; sodium acetate; sodium fumarate; sodium chloride; DL-leucine; polyethylene glycol; sodium oleate; sodium lauryl sulfate; and magnesium lauryl sulfate. Use of magnesium stearate as a lubricant is particularly preferred. In another aspect described herein, magnesium stearate is a preferred lubricant used, for example, to reduce friction between the equipment and granulated mixture during compression of tablet formulations. Such lubricants, if present, constitute about 0.1% to about 10%, preferably about 0.25% to about 5%, more preferably about 0.5% to about 2% of the total weight of the composition.

Glidants can be used to promote powder flow of a solid formulation. Suitable glidants include colloidal silicon dioxide, starch, talc, tribasic calcium phosphate, powdered cellulose and magnesium trisilicate. Colloidal silicon dioxide is particularly preferred. Such glidant, if present, constitutes about 0.05% to about 5%, more preferably about 0.1% to about 5%, more preferably about 0.25% to about 2% of the total weight of the composition.

The dispersible tablet compositions described herein can also include other excipients such as colorants, flavors and sweeteners. Suitable sweeteners include artificial sweeteners such as sucralose, aspartame, cyclamates, acesulfame potassium, saccharin or mixtures thereof; or sugars such as sucrose, fructose, dextrose or glucose. Sweeteners, if present, constitute about 0.01% to about 10%, preferably about 0.05% to about 5%, more preferably about 0.1% to 2% of the total weight of the formulation.

Any conventional, approved flavorants may be chosen so long as they do not materially affect the physical or chemical attributes of the active agent. Flavorants can include vanilla, strawberry, cherry, grape, wild berry, lemon, lime, orange, peppermint, spearmint, cinnamon, and any desired combination thereof. Flavorant, if present, constitutes about 0.01% to about 10%, preferably about 0.05% to about 5%, more preferably about 0.1% to 2% of the total weight of the formulation.

The tablet composition preferably includes a sweetener, more preferably both a flavorant and sweetener to mask the bitter taste deflazacort.

The tablets can be coated, for example with a non-functional cosmetic coating, dry powder compression coating, or uncoated. If the tablets are coated, a coating is preferably selected that does not interfere with dispersion of the tablet composition in water.

Compositions described herein can further comprise, for example, buffering agents.

In one aspect provided herein, a dispersible tablet comprises deflazacort, water-soluble diluent, a water swellable diluent, a superdisintegrant, a lubricant and a glidant, wherein the water-soluble diluent is mannitol, the water swellable diluent is mannitol, the lubricant is microcrystalline cellulose, and the glidant is sodium starch glycolate. In anotheraspect provided herein, the dispersible tablet comprises about 1% to about 10% deflazacort, about 25% to about 45% mannitol, about 30% to about 55% microcrystalline cellulose, about 5% to about 20% croscarmellose sodium and/or sodium starch glycolate, about 0.5% to about 2% magnesium stearate, and about 0.25% to about 2% colloidal silicon dioxide, wherein the ratio of microcrystalline cellulose to mannitol is about 1.2 to 1.65. In another aspect, the dispersible tablet further comprises at least one of a sweetener and a flavorant.

In one aspect, a process for manufacturing a dispersible tablet comprises forming a blend comprising deflazacort, a water-soluble diluent, a water swellable diluent, a lubricant, and a glidant and compressing the resultant blend into a tablet.

### EXAMPLES

The following examples illustrate aspects of the invention. The examples are not to be construed as limitations.

The deflazacort used in all of the examples below was in micronized form. 95% of the micronized particles of deflazacort had a particle size of less than about 10 μm. 90% of the micronized particles of deflazacort had a particle size of less than about 5 μm.

### Example 1

Compressed tablets were prepared with the formulae shown in Table 1:

TABLE 1

| | Formulation | | | | | |
|---|---|---|---|---|---|---|
| | Common | 1A | 1B | Common | 2A | 2B |
| | Blend | mg/ | mg/ | Blend | mg/ | mg/ |
| Ingredient | % (w/w) | unit | unit | % (w/w) | unit | unit |
| Deflazacort | 10.0 | 1.0 | 36.0 | 10.0 | 1.0 | 36.0 |
| Mannitol 100 SD | 38.0 | 3.8 | 136.8 | 33.0 | 3.3 | 118.8 |
| Microcrystalline cellulose 102 | 45.0 | 4.5 | 162.0 | 45.0 | 4.5 | 162.0 |
| Sodium starch Glycolate | 5.0 | 0.5 | 18.0 | 10.0 | 1.0 | 36.0 |
| Colloidal silicon dioxide | 1.0 | 0.1 | 3.6 | 1.0 | 0.1 | 3.6 |
| Magnesium Stearate | 1.0 | 0.1 | 3.6 | 1.0 | 0.1 | 3.6 |
| Total | 100 | 10.0 | 360.0 | 100 | 10.0 | 360.0 |

Tablets were produced as follows:
1. Deflazacort, colloidal silicon dioxide, sodium starch glycolate, and one third of the mannitol 100 SD were each sieved, introduced to a V-blender, and mixed.
2. The remaining portion of mannitol was sieved, added to the blender and mixed.
3. Microcyrstalline cellulose 102 was sieved, added to the blender after step 2, and mixed.
4. Magnesium stearate was sieved, added to the blender after step 3, and mixed.
5. The final blend from step 4 was compressed using 2.0 mm round concave tooling for the 1 mg tablets and using 10.0 mm round concave tooling for the 36 mg tablets.

The tablets produced as described above were tested for hardness, friability, and disintegration time. The disintegration tests in this Example and in the other Examples below were performed at 20° C. The analysis results are summarized in Table 2, below.

TABLE 2

| Sample | Tablet | Average | Friability | Disintegration |
|---|---|---|---|---|
| No. | Strength (mg) | Hardness (kP) | (%) | Time (seconds) |
| 1A | 1.0 | 2.6 | Failed test | 6 |
| 1B | 36.0 | 11.5 | 0.17 | 29 |
| 2A | 1.0 | 2.0 | Not tested | 5 |
| 2B | 36.0 | 10.6 | 0.16 | 43 |

For the first common blend, it was found deflazacort was difficult to sieve over a 30-mesh screen, so for blend 2 and all other examples below, deflazacort was sieved over a 18 mesh sieve prior to dry blending. For tablet samples 1A, the tablets clung to the inside of the friability tester drum, hampering them from tumbling. Those tablets failed the friability test due to capping of at least one tablet observed after the test. The mini tablets (1A and 2A) disintegrated in less than 10 seconds, compared to between 29 to 43 seconds for the 36 mg tablets made from the same blends.

### Example 2

Compressed tablets were prepared with the formulae shown in Table 3:

TABLE 3

| | Sample No. | | | | | |
|---|---|---|---|---|---|---|
| | 3 | 4 | 5 | 6 | 7 | 8 |
| Ingredient | mg/unit | mg/unit | mg/unit | mg/unit | mg/unit | mg/unit |
| Deflazacort | 36.0 | 36.0 | 36.0 | 36.0 | 36.0 | 36.0 |
| Mannitol 100 SD | 345.6 | 255.6 | 273.6 | 273.6 | 216.0 | 252.0 |
| Microcrystalline Cellulose 102 | 252.0 | 252.0 | 316.8 | 306.0 | 338.4 | 252.0 |
| Croscarmellose sodium | 72.0 | 82.8 | | 75.6 | | 82.8 |
| Xanthan gum | | | 3.6 | 14.4 | | |
| Sucralose | | | | | 3.6 | |
| Sodium starch glycolate | | 82.8 | 75.6 | | 116.6 | 82.8 |
| Colloidal silicon dioxide | 7.2 | 7.2 | 7.2 | 7.2 | 7.2 | 7.2 |
| Magnesium stearate | 7.2 | 3.6 | 7.2 | 7.2 | 7.2 | 7.2 |
| Total | 720.0 | 720.0 | 720.0 | 720.0 | 720.0 | 720.0 |

Tablets were produced as follows:
1. Deflazacort, colloidal silicon dioxide, croscarmellose or sodium starch glycolate, and ⅓ of the mannitol 100 SD were mixed in a V-blender.
2. The blend of step 1 was sieved and reintroduced to the blender.
3. The remaining Mannitol 100 SD was sieved, added to the blend of step 2 in the blender and mixed.
4. Microcrystalline cellulose 102 and, if present, sodium starch glycolate were sieved, added to the blender, and mixed.
5. Magnesium stearate was sieved, added to the blender, and mixed.
6. The resulting blend of step 5 was compressed to form compressed tablets using oval concave tooling or round flat face beveled edge tooling.

The tablets produced as described above were tested for hardness, friability, and disintegration time. The analysis results are summarized in Table 4, below.

TABLE 4

| | | Average | Friability | Disintegration |
|---|---|---|---|---|
| Sample No. | | Hardness (kP) | (%) | Time (seconds) |
| 3 | (oval) | 13.5 | 0.18 | 32 |
| 3 | (round) | 16.4 | 0.13 | 42 |
| 4 | (oval) | 12.6 | 0.28 | 38 |
| 5 | (oval) | 16.1 | Not tested | 76 |
| 5 | (round) | 15.4 | Not tested | 72 |
| 6 | (oval) | 14.5 | Not tested | >1380 |
| 7 | (round) | 16.8 | 0.01 | 48 |
| 8 | (round) | 16.2 | 0.01 | 56 |

All the tablets produced as described above were harder than those produced as in Example 1, above, and there were no issues with friability testing. Except the tablets from Sample No. 4, with 2% by weight xantham gum, disintegrated in under 3 minutes. Both the oval and round flat faced beveled edge shaped tablets had comparable friability, hardness, and disintegration times. All tablets disintegrated quickly, regardless of whether sodium starch glycolate, croscarmellose sodium, or a combination of the two were included as superdisintegrants.

### Example 3

Compressed tablets with 3 or 36 mg/unit of deflazacort were produced from three different common blends, containing 5% by weight deflazacort per blend, using different ratios of mannitol and microcrystalline cellulose, and different flavors, as shown in Table 5, below:

TABLE 5

| | Formulation | | | | | | | | |
|---|---|---|---|---|---|---|---|---|---|
| | Common | 9A | 9B | Common | 10A | 10B | Common | 11A | 11B |
| | Blend | mg/ | mg/ | Blend | mg/ | mg/ | Blend | mg/ | mg/ |
| Ingredient | % (w/w) | unit | unit | % (w/w) | unit | unit | % (w/w) | unit | unit |
| Deflazacort | 5.0 | 36.0 | 3.0 | 5.0 | 36.0 | 3.0 | 5.0 | 3.0 | 36.0 |
| Mannitol 100 | 30.6 | 220.3 | 18.4 | 46.5 | 334.8 | 18.4 | 35.0 | 21.0 | 252.0 |
| SD | | | | | | | | | |
| Microcrystalline | 46.5 | 334.8 | 27.9 | 30.6 | 220.3 | 27.9 | 35.0 | 21.0 | 252.0 |
| Cellulose 102 | | | | | | | | | |
| Croscarmellose | | | | | | | 11.5 | 6.9 | 82.8 |
| sodium | | | | | | | | | |
| Strawberry | | | | 0.5 | 3.6 | 0.3 | | | |
| flavor | | | | | | | | | |
| Cherry flavor | 0.5 | 3.6 | 0.3 | | | | | | |
| Sucralose | 0.5 | 3.6 | 0.3 | 0.5 | 3.6 | 0.3 | | | |
| Sodium starch | 15.5 | 111.6 | 9.3 | 15.5 | 116.6 | 9.3 | 11.5 | 6.9 | 82.8 |
| Glycolate | | | | | | | | | |
| Colloidal silicon | 0.4 | 2.9 | 0.2 | 0.4 | 2.9 | 0.2 | 1.0 | 0.6 | 7.2 |
| Dioxide | | | | | | | | | |
| Magnesium Stearate | 1.0 | 7.2 | 0.6 | 1.0 | 7.2 | 0.6 | 1.0 | 0.6 | 7.2 |
| Total | 100 | 720.0 | 60.0 | 100 | 720.0 | 60.0 | 100 | 60.0 | 720.0 |

Tablets were produced using the same process as in Example 1. 5.0 mm round 3 mg strength compressed tablets, and 13 mm round flat faced beveled edge 36 mg strength tablets were produced. Results of testing the tablets are shown in Table 6, below:

TABLE 6

| | Tablet | Average | Friability | Disintegration |
|---|---|---|---|---|
| Sample No. | Strength (mg) | Hardness (kP) | (%) | Time (seconds) |
| 9A | 36 | 12.8 | 0.15 | 49 |
| 9B | 3 | 4.6 | 0.02 | 37 |
| 10A | 36 | 14.3 | 0.27 | 50 |
| 10B | 3 | 3.6 | 0.15 | 38 |
| 11A | 3 | 3.4 | 0.08 | 23 |
| 11B | 36 | 14.2 | 0.23 | 47 |

All of the tablets tested disintegrated in less than a minute. Of the tablets tested, tablets 9A and 9B, with a ratio of mannitol to microcrystalline cellulose of 30.6 to 46.5 had the lowest friability.

### Example 4

Compressed tablets with 3 or 36 mg/unit of deflazacort were produced from three different common blends, containing 6% by weight deflazacort per blend, as shown in Table 7:

TABLE 7

| | Formulation | | | | | | | | |
|---|---|---|---|---|---|---|---|---|---|
| | | 12A | 12B | | 13A | 13B | | 14A | 14B |
| | Blend | mg/ | mg/ | Blend | mg/ | mg/ | Blend | mg/ | mg/ |
| Ingredient | % w/w | unit | unit | % w/w | unit | unit | % w/w | unit | unit |
| Deflazacort | 6.0 | 3.0 | 36.0 | 6.0 | 3.0 | 6.0 | 6.0 | 3.0 | 6.0 |
| Mannitol 100 SD | 30 | 15 | 180 | 30 | 15 | 180 | 30 | 15 | 180 |
| Microcrystalline | 51.5 | 25.75 | 51.25 | 51.25 | 220.3 | 27.9 | 51.0 | 21.0 | 252.0 |
| cellulose 102 | | | | | | | | | |
| Strawberry flavor | 0.5 | 0.25 | 3.0 | 0.5 | 0.25 | 3.0 | 0.5 | 0.25 | 3.0 |
| Sucralose | 0.5 | 0.25 | 3.0 | 0.5 | 0.25 | 3.0 | 0.5 | 0.25 | 3.0 |
| Sodium starch | 10 | 5.0 | 60.0 | 10 | 5.0 | 60.0 | 10 | 5.0 | 60.0 |
| glycolate | | | | | | | | | |
| Colloidal silicon | 0.5 | 0.25 | 3.0 | 0.5 | 0.25 | 3.0 | 0.5 | 0.25 | 3.0 |
| dioxide | | | | | | | | | |
| Magnesium Stearate | 1.0 | 0.5 | 6.0 | 1.25 | 0.63 | 7.5 | 1.5 | 0.57 | 9.0 |
| Total | 100 | 720.0 | 60.0 | 100 | 720.0 | 60.0 | 100 | 60.0 | 720.0 |

Tablets 12A and 12B were prepared as follows:
1. Sieving about half of the colloidal silicon dioxide, about 1/12 of the mannitol 100 SD and about 1/12 of the microcrystalline cellulose and mixing in a blender,
2. Sieving the deflazacort and adding that to the resulting mixture from step 1, and mixing,
3. Sieving and adding strawberry, sucralose and about ⅓ of the mannitol, and mixing,
4. Sieving and adding the remaining colloidal silicon dioxide, sodium starch glycolate, and remaining microcrystalline cellulose, and mixing,
5. Sieving and adding the magnesium stearate and mixing.
6. Preparing compressed tablets of the resulting mixture from 5, using 5.0 mm round concave tooling for the 3.0 mg tablets and 13.0 mm round flat face tooling for the 6.0 mg tablets.

Tablets 13A and 13B were prepared in a similar way to 12A and 12B except that deflazacort was mixed and blended with the excipients in step 1 prior to sieving.

Tablets 14A and 14B were prepared as follows:
1. Introducing about 1/10 of the microcrystalline cellulose to a blender.
2. Mixing the colloidal silicon dioxide, deflazacort, and about 1/10 of the mannitol 100 SD in a bag before adding to the blender in step 1 and blending
3. Sieving the mixture from step 2, and reintroducing to a blender
4. Sieving a mixture of strawberry, sucralose, about ⅓ of mannitol 100 SD and about 1/10 of microcrystalline cellulose, adding to the blender in step 3, and mixing,
5. Sieving the remaining mannitol SD and about 1/10 of microcrystalline cellulose, adding to the blender, and mixing,
6. Sieving sodium starch glycolate the remaining microcrystalline cellulose, adding to the blender from step 5, and mixing,
7. Sieving the magnesium stearate, adding it to the blender, and mixing, and
8. Preparing tablets of 50 and 600 mg of the resulting mixture, as described for Samples 10A and 10B.

The tablets were tested for hardness, friability, and disintegration time, as shown in Table 8:

TABLE 8

| | Tablet | Average | Friability | Disintegration |
|---|---|---|---|---|
| Sample No. | Strength (mg) | Hardness (kP) | (%) | Time (seconds) |
| 12A | 3 | 4.0 | 0.06 | 41 |
| 12B | 36 | 0.7 | 0.14 | 30 |
| 13A | 3 | 3.2 | 0.6 | 20 |
| 13B | 36 | 14.0 | 0.08 | 40 |
| 14A | 3 | 3.8 | 0.03 | 50 |
| 14B | 36 | 14.7 | 0.02 | 45 |

All of the tablets tested as described in Table 8 disintegrated in less than 1 minute and had low friability. Samples 14A and 14B had the lowest friability.

### Example 4

Round flat faced compressed tablets with the same composition and size as 14B, above (36 mg deflazacort), prepared with wild berry flavor instead of strawberry were tested for stability in bottles with desiccant, under different temperatures and conditions. The bottles were stored at 25° C. and 60% relative humidity, and at 40° C. and 75% relative humidity for 3 months. The dissolution profile was tested by placing each tablet in 50mM sodium phosphate buffer, pH 6.8 with 0.1% sodium lauryl sulfate. Three tablets were removed at each of several different time points. The average of each set of tablets are shown in Table 7, below:

TABLE 7

| | | 25° C. | 25° C. | 40° C. | 40° C. | 40° C. |
|---|---|---|---|---|---|---|
| Time | | 2 months | 3 months | 2 weeks | 2 months | 3 months |
| (min) | T = 0 | % release | % release | % release | % release | % release |
| 5 | 85 | 86 | 91 | 83 | 84 | 94 |
| 10 | 91 | 93 | 96 | 89 | 93 | 98 |
| 15 | 92 | 94 | 97 | 91 | 94 | 98 |
| 30 | 94 | 95 | 97 | 94 | 95 | 98 |
| 45 | 94 | 95 | 97 | 95 | 95 | 98 |

No significant difference in dissolution profile was observed after the three month stability study. All tablets also showed less than 0.10 total impurities at each time point. Similar results were obtained from tablets stored under the same conditions in blisters for three months.

Having fully described the subject matter of the claims, it will be understood by those having ordinary skill in the art that the same can be performed within a wide range of equivalents without affecting the scope of the subject matter or particular aspects described herein.

We claim:

1. A dispersible tablet comprising: deflazacort, a water-soluble diluent, a water swellable diluent and a superdisintegrant, wherein the tablet disintegrates within 3 minutes when added to water.

2. The dispersible tablet of claim 1, wherein the deflazacort is about 1% to about 20% by weight of the tablet.

3. The dispersible tablet of claim 1, wherein the water-soluble diluent is selected from the group consisting of mannitol, lactose, xylitol, sorbitol, calcium sulfate dihydrate, inositol, dextrin, calcium sulfate anhydrate, fructose, kaolin, sucrose, lactitol, dextrates, sodium chloride, and dextrose or a combination thereof.

4. The dispersible tablet of claim 1, wherein the water-soluble diluent is mannitol.

5. The dispersible tablet of claim 1, wherein the water-soluble diluent is about 10% to about 60% by weight of the tablet.

6. The dispersible tablet of claim 1, wherein the water swellable diluent is selected from the group consisting of microcrystalline cellulose, pregelatinized starch, starch, powdered cellulose, silicified microcrystalline cellulose, dibasic calcium phosphate dihydrate, calcium phosphate, calcium carbonate, hydroxypropylcellulose, hydroxyethylcellulose, and hydroxypropyl methylcellulose or a combination thereof.

7. The dispersible tablet of claim 1, wherein the water swellable diluent is microcrystalline cellulose

8. The dispersible tablet of claim 1, wherein the water swellable diluent is 10% to about 70% by weight of the tablet

9. The dispersible tablet of claim 1, wherein the ratio of water-soluble diluent to water swellable diluent is 0.5 to about 1.8.

10. The dispersible tablet of claim 1, wherein the superdisintegrant is selected from the group consisting of croscarmellose sodium, sodium starch glycolate, and crospovidone or a combination thereof.

11. The dispersible tablet of claim 1, wherein the superdisintegrant is about 0.5% to 20% by weight of the tablet.

12. The dispersible tablet of claim 1, further comprising a lubricant.

13. The dispersible tablet of claim 12, wherein the lubricant is magnesium stearate.

14. The dispersible tablet of claim 12, wherein the lubricant is about 0.1% to about 10%, by weight of the tablet.

15. The dispersible tablet of claim 1, further comprising a glidant.

16. The dispersible tablet of claim 15, wherein the glidant is colloidal silicon dioxide.

17. The dispersible tablet of claim 15, wherein the glidant is about 0.05% to about 5% by weight of the tablet.

18. The dispersible tablet of claim 1, further comprising a sweetener.

19. The dispersible tablet of claim 1, further comprising a flavoring agent.

20. A process for preparing the dispersible tablet of deflazacort of claim 1, comprising blending a composition comprising the deflazacort, the water-soluble diluent, the water swellable diluent, the superdisintegrant, a lubricant, and a glidant to form an admixture, and compressing the admixture to form a tablet.

21. The process of claim 20, wherein the composition further comprises a sweetener.

22. The process of claim 20, wherein the composition further comprises a flavoring agent.

23. A method of treatment of muscular dystrophy, comprising dispersing a dispersible tablet of claim 1 in water and administering it to a subject in need thereof.

24. The method of treatment of claim 23, wherein the muscular dystrophy is selected from Duchenne muscular dystrophy or Limb-girdle muscular dystrophy.
